(19) United States
(12) Patent Application Publication
(10) Pub. No.: US 2010/0087751 A1
(43) Pub. Date: Apr. 8, 2010

(54) ACOUSTIC REFLECTOMETRY INSTRUMENT AND METHOD

(76) Inventor: David P. Warner, Prairie Village, KS (US)

Correspondence Address: HUSCH BLACKWELL SANDERS LLP, 4801 Main Street, Suite 1000, KANSAS CITY, MO, 64112, US

(21) Appl. No.: 12/244,987

(22) Filed: Oct. 3, 2008

(51) Int. Cl. A61B 5/00

(52) U.S. Cl. 600/559

(57) ABSTRACT

An acoustic reflectometry instrument and method for ascertaining fluid presence in an ear. Drive side normalization involves normalizing the speaker to adjust its output to compensate for non-linearity over a frequency range used for acoustic reflectometry measurement of the ear. The microphone is normalized to compensate for system non-linearity. Measurement involves repetition a selected number of times until the spectral gradient angle is within a specified range for each repetition to provide validation of the measurement. For enhanced reliability, various sources of error are tested.

### CROSS-REFERENCE TO RELATED APPLICATIONS

None.

### FIELD OF THE INVENTION

This invention relates generally to the field of acoustic reflectometry and deals more particularly with a method and apparatus making use of acoustic reflectometry for evaluation of the ear to detect the presence of fluid which may be an indication of Otitis Media.

### BACKGROUND OF THE INVENTION

Acoustic reflectometry is known to be useful in detecting the presence of fluid in the middle ear cavity. Excessive fluid levels may be an indication of Otitis Media or other afflictions. Acoustic reflectometry techniques typically involve propagating an audio signal at a number of frequencies through the ear canal. The reflected energy may be analyzed using known processes to determine the mechanical resonance characteristics of the ear drum. The shape of the frequency response curve is used to determine a spectral gradient angle which is a measurement of the characteristics of a null in the spectral curve.

In healthy ears, the ear drum has ample freedom of motion because the middle ear cavity is not under pressure. Accordingly, the resonance curve is shallow and the spectral gradient angle has a low value. Conversely, when fluid is present in the middle ear space, it restricts the motion of the ear drum. Then, a sharp resonance curve results along with a high spectral gradient value. Thus, the spectral gradient angle can be used to diagnose the likely presence or absence of fluid in the middle ear cavity and the relative risk of Otitis Media.

While instruments have been developed and used successfully based on acoustic reflectometry, they have not been wholly without problems. The relatively high cost of the instruments has been a notable disadvantage. In particular, expensive microphones and speakers have been required in order to provide acceptable levels of accuracy and reliability. Because of the need for costly components, it has been impractical to provide instruments that are inexpensive enough to be made available to ordinary consumers as opposed to medical professionals.

### SUMMARY OF THE INVENTION

The present invention is directed to an acoustic reflectometry instrument and method employing improved techniques to increase the accuracy and reliability of the measurements while allowing for relatively low cost components.

In accordance with one aspect of the invention, drive side normalization is used to normalize the speaker such that its power level is substantially equal at all frequencies, thereby compensating for non-linearity of the frequency response. Accordingly, and when used with a normalization of the microphone and the calibration of the instrument, a lower cost is achieved because there is no need for a high cost speaker as has been required in the past.

In accordance with another aspect of the invention, the accuracy of the measurement is enhanced by repeating each measurement a selected number of times and confirming that each repetition results in a spectral gradient angle which does not depart unduly from the other repetitions. A time limit to complete all repetitions may be imposed for even more rigorous validation of the measurements. Various sources of error may also be investigated to provide greater assurance that the measurements are valid, thereby enhancing the reliability and accuracy of the instrument.

Other and further objects of the invention, together with the features of novelty appurtenant thereto, will appear in the course of the following description.

### BRIEF DESCRIPTION OF THE SEVERAL VIEWS OF THE DRAWING

In the accompanying drawings, which form a part of the specification and are to be read in conjunction therewith and in which like reference numerals are used to indicate like or similar parts in the various views:

FIG. 1 is a perspective view of an acoustic reflectometry instrument constructed according to a preferred embodiment of the present invention;

FIG. 2 is a schematic diagram of the principal components of the acoustic reflectometry instrument of FIG. 1;

FIG. 3 is a block diagram illustrating a preferred normalization and calibration process that may be used in accordance with the invention;

FIG. 4 is a flow chart of a preferred measurement process that may be used in accordance with the invention; and

FIG. 5 is a flow chart of a preferred wake and sleep process conducted in accordance with the invention.

### DETAILED DESCRIPTION OF THE INVENTION

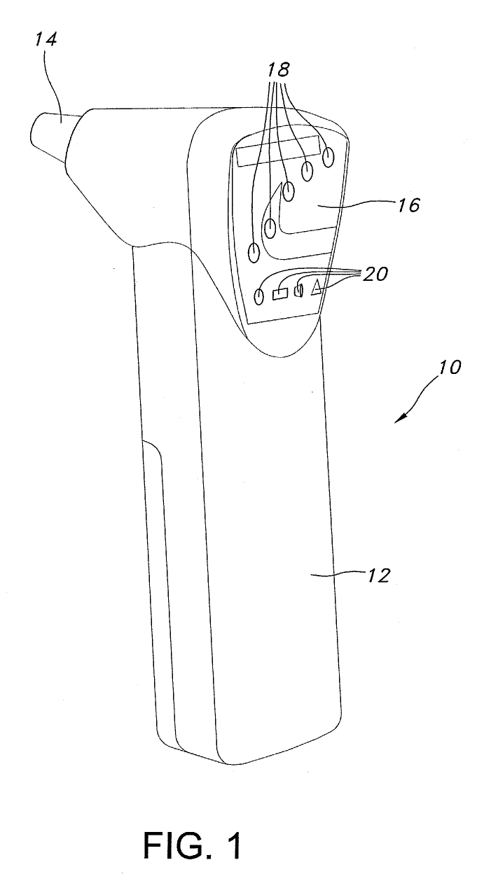

Referring now to the drawings in more detail and initially to FIG. 1, the present invention is directed to the use of acoustic reflectometry for evaluation and diagnosis of the ear. The invention may be implemented using an instrument which is generally identified by numeral 10 and which may be a handheld device having a handle 12. The head end of the instrument 10 is provided with a tip 14 having a size and shape to be inserted into an ear that is to be diagnosed. The head of the instrument may also be provided with a panel 16 having various control components and displays which may include a number of LED indicators 18 and other indicators 20.

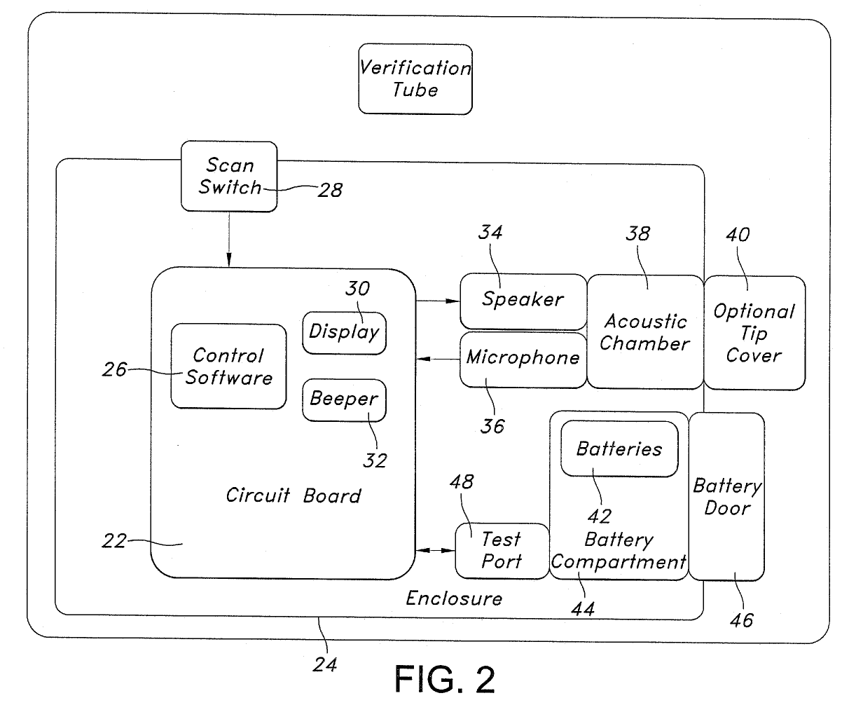

FIG. 2 depicts diagrammatically the principal components of the instrument 10. A circuit board 22 within the enclosure or housing 24 of the instrument controls its operation using control software 26. A scan switch 28 may be activated to initiate a measurement. A display 30 and beeper 32 or other audible device may be included and selectively activated by the control software 26. A speaker 34 and microphone 36 are associated with an acoustic chamber 38 which connects with the tip 14. The tip 14 may be optionally covered by a removable tip cover 40.

Power is supplied by conventional batteries 42 contained in a battery compartment 44 accessible through a door 46 on the housing 24. A test port 48 is provided to allow access through the battery compartment 44 for testing and trouble shooting purposes.

In accordance with the embodiment of the invention shown in FIGS. 1 and 2, incident sound waves from the speaker 34 are propagated through the acoustic chamber 38 and tip 14 into the ear canal to the tympanic membrane. The incident waves are propagated at a variety of frequencies in a frequency range that includes the resonant frequency of the tympanic membrane. Some of the incident waves are reflected back through the tip 14 and acoustic chamber 38 to the microphone 36 which obtains the vector sum of the incident and reflected waves. An acoustic reflective curve for the frequency spectrum is calculated, and the null value of the curve is determined. This allows for a measurement of the spectral gradient angle of the null of the spectral curve, providing a basis for determining the presence of fluid in the ear cavity and allowing diagnosis of one or more conditions of the ear (principally, the relative risk of Otitis Media).

Instrument accuracy depends on the speaker 34 putting out substantially the same power level over the full range of frequencies that are propagated. The present invention provides for normalizing the drive levels so that each frequency outputs a sound pressure level equal to an average pressure level obtained over the frequency within a predetermined range. For example, known output pressure levels from a device known to be accurate may be obtained and averaged over the frequency range to be used. The speaker drive is thus set to compensate for the variance from what the speaker output should be ideally. This procedure may be referred to as drive side normalization.

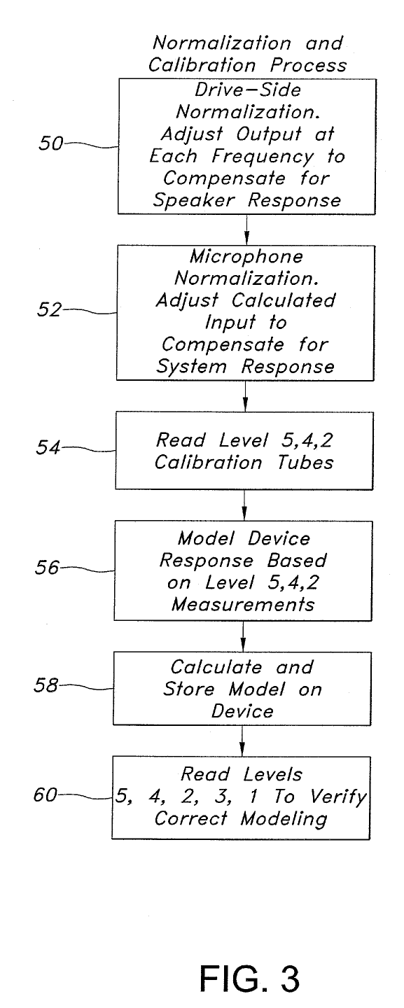

The drive side normalization is effected in block 50 in FIG. 3. The output is adjusted at each frequency to compensate for the nonlinear speaker response. By carrying out drive side normalization in this manner, a relatively inexpensive speaker can be used because the speaker is not required to provide a flat frequency response. With continued reference to FIG. 3, the normalization and calibration process includes microphone normalization in block 52 wherein the input is adjusted to compensate for the nonlinearity of the system response. In block 54, a plurality of calibration tubes are used having known expected responses, and the actual responses of the tubes are obtained during the calibration process. In block 56, the differences between the expected calibration tube responses and the actual responses are compared and used to construct a model which is stored as indicated at block 58. The accuracy of the calibration is verified in block 60 by comparing readings of the calibration tubes indicated by the model with the known expected readings.

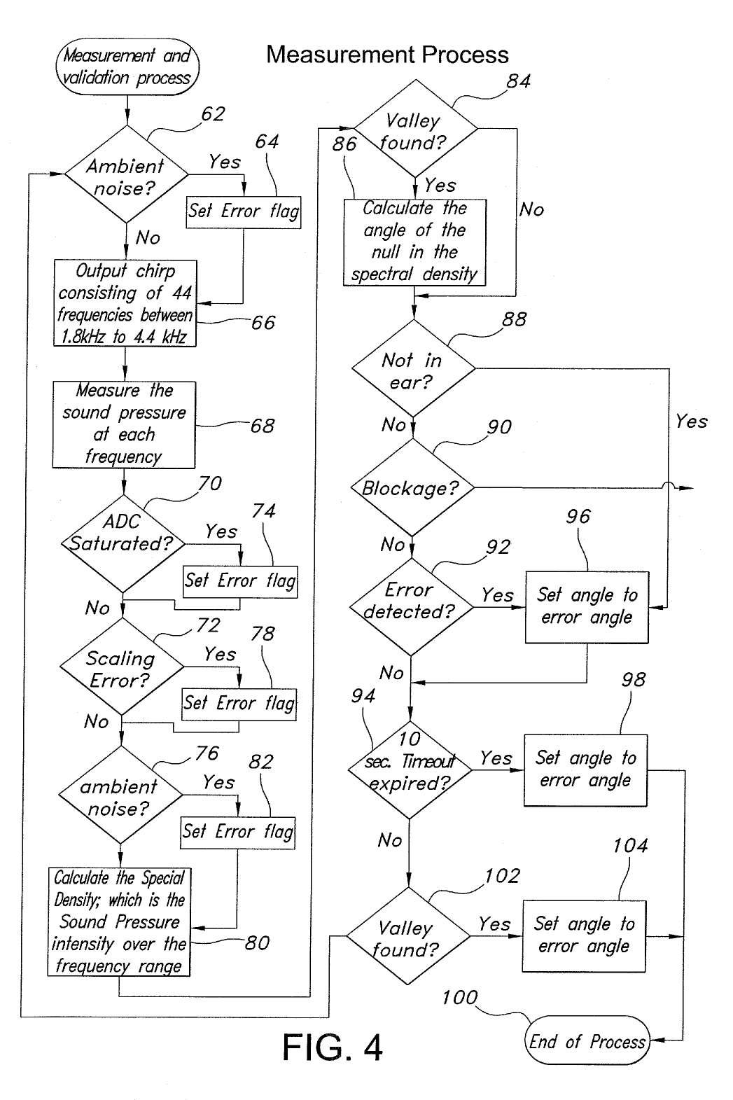

FIG. 4 depicts in flow chart form the process the instrument I 0 uses to take measurements indicative of the health of the ear and to validate the measurements.

First, a determination is made at block 62 as to the presence of ambient noise. If ambient noise is present, an error flag is set in block 64 before block 66 is entered. If there is no ambient noise, block 66 is entered directly from block 62. In block 66, an output “chirp” is generated by the speaker 34. For example, the chirp may consist of a sequence of tones that are generated at each of 44 different frequencies within a selected frequency range (which may be from 1.8 kHz to 4.4 kHz. Eight cycles of each frequency (“ramp up” cycles) may be used to allow stabilization of the acoustic system before a measurement is made. Four cycles (“ramp down” cycles) may be used to reduce resonance.

The sound pressure or intensity is measured using the microphone 36. If there is no ADC saturation as determined in block 70, block 72 is entered. If there is ADC saturation, block 74 is entered to set an error flag, and block 72 is then entered. If there is no scaling error detected in block 72, block 76 is entered. If there is a scaling error, block 78 is entered to set an error flag before block 76 entered. If there is an absence of ambient noise as determined in block 76, block 80 is entered. If ambient noise is detected, block 82 sets an error flag before block 80 is entered. From the measurements of the same pressures at each frequency (block 68), the sound pressure is plotted over the frequency range to provide a calculation of the spectral density in block 80. Then, a determination is made in block 84 as to the detection of a valley. If there is a valley, the process moves to block 86. In block 86, the angle of the null in the spectral density plot is determined to provide the spectral gradient angle. The next step is carried out in block 88. If a valley is not detected, block 86 is bypassed and block 88 is entered directly from block 84. If it is determined in block 88 that the instrument is not properly in the ear, block 90 is entered to determine if there is a blockage. If there is not, block 92 is entered to determine if there is an error. In order to minimize the potential for error, a successful measurement requires four chirps to result in spectral gradient angles within a selected range and time (such as within + or −5° and within 10 seconds, for example), and also without error conditions present. If there is not a successful measurement satisfying these requirements, the process is repeated until there is a successful measurement. Thus, in block 94 a determination is made as to whether there has been expiration of a 10 second timeout period. If it is determined in block 88 that the instrument is not properly in the ear or if an error is detected in block 92, block 96 is entered to set the angle to an error angle, and block 94 is then entered.

If it is determined in block 94 that the 10 second timeout period has elapsed, block 96 is entered to display a “try again” status of the instrument, and then the process is ended in block 100. If the 10 second timeout period has not expired, block 102 is entered from block 94 and a determination is made as to whether two good angles have been generated within 10°. If not, block 62 is entered from block 102 and the process is repeated. If two good angles within 10° are detected in block 102, block 104 is entered and a display of the risk level associated with the detected angle is generated prior to ending the process in block 100.

Generally, lower spectral gradient angles indicate healthier ears and a lesser risk of Otitis Media. One aspect of the present invention contemplates that the risk of Otitis Media may be categorized into different levels depending on the spectral gradient angle. By way of example, five risk levels may be established, with a spectral gradient angle less than 49° falling into a low risk level of 5, angles equal to or greater than 49° and equal to or less than 59° falling into risk level 4, angels greater than 59° but less than or equal to 69° falling into risk level 3, angles greater than 69° but less than or equal to 89° falling into risk level 2, and angles greater than 89° falling into the highest risk level of one. For each successful measurement, the risk level (block 104) in accordance with the foregoing may be displayed on the panel 16, as by energizing appropriate LEDs 18.

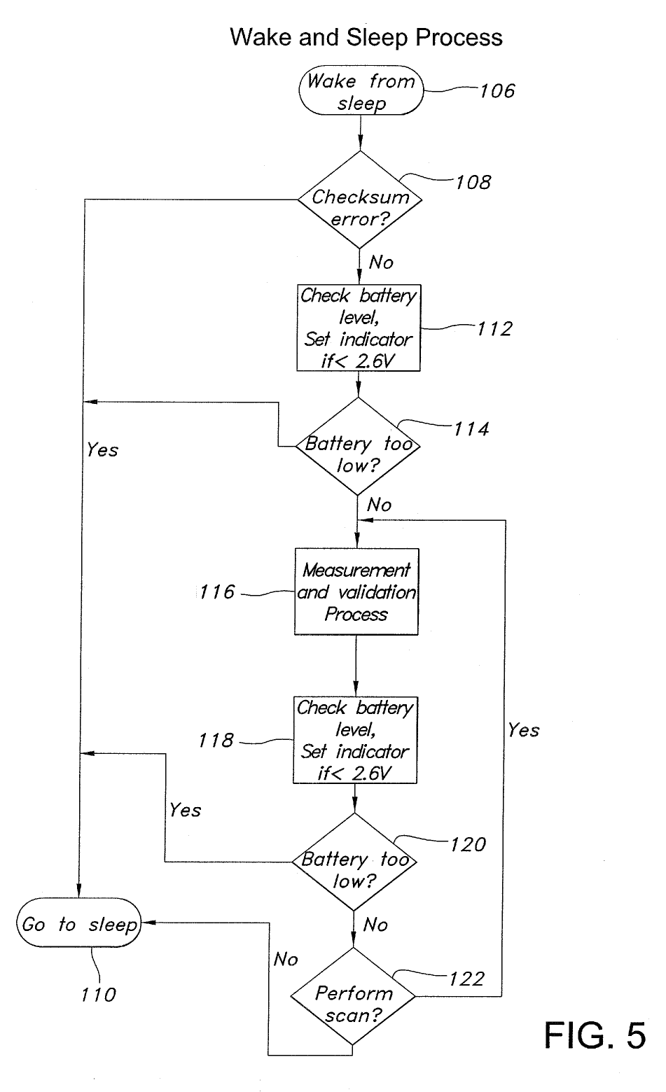

FIG. 5 depicts a wake and sleep process that may be included. When the instrument “wakes” from a sleeping condition in block 106, block 108 is entered to determine if a check sum error has occurred. If it has, block 110 is entered and the instrument reverts to the sleep mode. If there is no check sum error, the battery level is checked in block 112 and a low battery indicator is set if the battery level is less than a predetermined value (2.6 volts, for example). If the battery is in an unduly low condition, block 110 is entered and the instrument reverts to the sleep mode. If the battery is not unduly low, block 116 is entered and the measurement and the validation process depicted in FIG. 4 is carried out. Then, the battery level is again checked in block 118 and an indicator is set if the battery is unduly low. If the battery is unduly low as indicated in block 120, block 110 is entered. If the battery is not unduly low, block 122 is entered and a performed scan determination is made. If the performed scan determination is negative, block 110 is entered. Conversely, if the performed scan determination is positive, block 116 is entered.

The instrument 10 may have a variety of modes of operation and may provide a variety of output signals and indications. For example, there may be a standby mode in which all visual and audio indicators are inactive. A scan mode may be entered when the scan switch 28 is activated. In the scan mode, all of the LED indicators 18 may be briefly activated. Also, self-tests may be conducted. Failure of a self-test generates a fault condition and entry into the error mode. When the scan mode is entered, a beep or other audible sound may be generated by the beeper 32 or other audio device. If the battery condition is unduly low, one of the indicators 20 may be energized to display a low battery indication. If the self-tests are completed successfully in the scan mode, the measurement process is initiated. A short beep or other signal may be provided between each chirp. During the measurement process, the LEDs 18 may be energized one at a time in sequence, with one sequence occurring for each chirp. At the end of a successful measurement process, the complete mode may be entered.

In the complete mode, a lengthy beep or other distinctive signal may be given to indicate a successful measurement. The Otitis Media risk level (1-5) may be displayed by energizing the LEDs 18 in a manner corresponding to the measured risk level. While the instrument is in the complete mode, the scan switch 28 may be activated to enter the scan mode again. After five seconds or some other selected time in the complete mode has elapsed without the scan switch having been activated, the instrument goes into the standby mode.

A fault condition or error results in the instrument entering the error mode. Three beeps or some other signal may then be generated as an indication of the error. If the error is due to failure of the self-test, a device error may be signaled audibly or on the panel 16 visually. If the error is due to an unsuccessful measurement, a visual display on panel 16 or an audible or other signal may be provided to identify the cause of the error, and a try again indication may be displayed on panel 16. When five seconds or some other selected time has elapsed, the instrument goes into the standby mode.

While the tip 14 may be removable and replaceable, it may also be an integral part of the instrument 10. In this case, removable tip covers such as the cover 40 may be provided so that after each use, the tip cover can be removed and replaced by an unused cover.

From the foregoing it will be seen that this invention is one well adapted to attain all ends and objects hereinabove set forth together with the other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth or shown in the accompanying drawings is to be interpreted as illustrative, and not in a limiting sense.

What is claimed is:

1. A process for adjusting an acoustic reflectometry device having a speaker for transmitting sound waves at different frequencies into an ear and a microphone for receiving reflected sound waves from the ear, said process comprising:
adjusting the speaker in a manner to provide a sound pressure level output at each of said frequencies substantially equal to an average pressure level obtained from a source known to be accurate at said frequencies;
adjusting the microphone in a manner to provide an input at each of said frequencies adjusted to compensate for non-linearity of the response of the device; and
using calibration devices with known responses to calibrate the device over a range of frequencies encompassing each of said different frequencies.

2. A process for effecting measurement of the acoustical reflectometry of an ear, comprising:
(a) propagating into the ear an acoustic tone at each of a plurality of different frequencies;
(b) measuring the acoustic intensity resulting from each of said tones;
(c) determining the spectral density of the measurements made in step (b);
(d) determining a spectral gradient angle from the spectral density determined in step (c);
(e) repeating steps (a)-(d) a preselected number of times;
(f) declaring a successful measurement if the spectral gradient angle determined for each of said preselected number of times is within a predetermined angular range; and
(g) determining a health condition of the near based on a successful measurement.

3. A process as set forth in claim 2, including the step of negating a successful measurement unless steps (a)-(d) are repeated said preselected number of times within a preselected time period.

4. A process as set forth in claim 2, including the step of displaying an indication of said health condition.

5. A process as set forth in claim 2, including repeating step (a) a preselected number of times before effecting step (b).

6. Apparatus for measuring the acoustical reflectometry of an ear, comprising:
a housing having a tip for insertion into the ear;
a speaker in said housing operable to propagate to said tip and into the ear an acoustic tone at each of a plurality of different frequencies, and to repeat propagation of each of said tones a preselected number of times;
a microphone in said housing operable to receive and measure the acoustic intensity resulting from each of said tones;
means for providing, from measurements of the acoustic intensity resulting from each of said tones, a spectral gradient angle of a spectral density of said measurements; and
means for determining a health condition of the ear if said spectral gradient angle is within a selected range for each of said preselected number of times.
